SEQUENCE LISTING (50 sequences)

SEQ ID NO: 1 - protein, 1560 aa, Homo sapiens [sequence omitted]

SEQ ID NO: 2 - DNA, 5682 bp, Homo sapiens
gcgcggccagagcggccggggacaggctccgaggcaggcccgacccgcctccccggcgccgccgtggctcgacggagaccagctaggctggcccccaagaggaccctttccaagtccccagctgggggccctgtgtagacctggagtggacacgcccctccttcccttcatgattcgtttgtagcgcagtggcgatggatgatgaggatgggagatgcttactagacgtgatttgtgacccacaggccctcaatgacttcttgcatggatccgagaagcttgacagtgatgacctcctggataatcccggggaggcccaaagtgccttctatgaaggtcctgggctccatgtgcaagaagcttccggcaaccacctgaacccagagcccaaccagccggcccccagtgtggacctagacttcctggaagatgacatcctgggctctcctgcgacagggggcggcggcgggggcagtgggggcgctgaccagccctgtgacatcctccagcagagcctccaagaggccaacatcacggagcagacgctggaggccgaggctgagctggacctgggtcccttccagctgcccaccctgcagcctgcggatggcggggcaggcccgacgggcgctggaggggcagcggccgtggctgcggggccccaagccctcttcccaggcagcactgacctgctggggctgcagggcccgcccaccgtgctgacccaccaggccctggtgccgccccaggacgtggtcaacaaggccctgagtgtgcagcccttcctgcagcctgtgggcctgggcaatgtgacactgcagcccatcccgggcctccaaggcctgcccaatggcagccctgggggtgccacggcggccacgctgggcctggcgcccatccaggtggtgggccagcccgtcatggcgctcaacacgcccacctcccagctcctggccaagcaggtgcccgtcagcggctacctggcctcggcggctggcccctcggagcccgtgacgctggcgtcggccggtgtctcgccacagggggctggcctggtcatccagaagaacctctcggccgctgtggccaccacgctcaatgggaactctgtgttcggaggcgcgggggccgcctcggctcccaccgggacgccctcgggacagccgctggcggtggccccaggcctcggctcgtcgccactggtcccggcgcccaacgtgatcctgcatcgcacacccacgcccatccagcccaagcccgcgggggtgctgccgcccaagctctaccagctgacgcccaagccgtttgcgcccgcgggcgccacgctcaccatccagggcgagccgggggcgctcccgcagcagcccaaggccccgcagaacctgacgttcatggcggcggggaaggcgggccagaacgtggtgctgtcgggcttccccgcgcctgcgctgcaagcgaacgtcttcaagcagccaccggccaccaccaccggagcggccccgccgcagccccccggggccctgagcaaacccatgagcgtccacctcctgaaccaaggcagcagcatcgtcatccccgcccagcacatgctgccgggccagaaccagttcctactgcctggcgccccggcggtccagctcccgcagcagctctcagccctgccggccaacgtgggcgggcagatcctggcggccgctgccccccacacaggtggacagctcatcgcgaaccccatcctcacaaaccagaacctggcgggcccactgagcctgggccccgtgttggccccccactccggggcccacagcgcgcacatcctctccgccgctcccatccaggtgggccagcctgcgctcttccagatgcccgtgtcgctggcggcgggcagcctgcccacgcagagccagccagcgcccgccgggccggccgccaccactgtcctccagggggtcaccctgccccccagcgccgtggccatgctcaacacccccgacggcctggtgcagccggccacccctgccgctgccaccggggaggccgcgcctgtcctcacggtgcagcctgccccccaggcgccccccgcggtcagcacacccctgcccctgggcctccagcagccgcaggcgcagcagcccccgcaggcccccaccccacaggccgccgccccgcctcaggccaccaccccccagcccagccctggcctggcgtctagcccggagaagatcgtcctggggcagccgccctctgccacccccacggccatcctcactcaggactccctgcagatgttcctgccccaggagaggagccagcagcccctctccgcagagggcccccacctctccgtgcctgcctcggtcatagtcagcgccccgcctcccgcccaagacccagccccagccacccccgtcgccaaaggagctggcctcggccctcaggcccccgacagccaggcttccccggctccggccccccagatcccggcagcggctccgctgaagggcccaggcccctcttcgtccccgtcactacctcaccaggcccctctgggggacagcccccacctgccctccccacaccccacccggcccccttcccgcccaccctcccggccacagagtgtgtcccgccctccctcagagccacccttgcacccttgccccccaccccaggcccccccaactctgcctggcatctttgtcatccaaaaccagctaggcgttcccccgcctgccagcaacccggcccctactgccccaggcccgccgcagccgcctctccgcccccagtcccagccgcctgagggaccgctgcccccagccccccacctccctccatcctccacctcctctgctgtggcctcctcctctgagacgtcctccaggttgccagcccctacgccatccgacttccagctccagttcccacccagccaggggccccacaagtcccccactccccctccaaccctccacctggtccctgagccggcagcaccccccccaccgcctcctcggaccttccagatggtgaccacccccttcccagcgctgccccagccgaaggctcttctcgagagatttcaccaggtgccgtccggaatcatcctccagaacaaggctgggggggcccctgccgccccgcagacctccaccagcctggggcccctcaccagccccgctgcgtctgtgctggtcagtgggcaggccccatctgggacccccactgcccccagccacgcccccgccccggcacccatggccgccacaggcctccctcctctgcttccagccgagaacaaggcttttgccagcaacctcccgaccctgaatgtggccaaggccgcttcctccgggccagggaagccctccgggctgcagtatgagagcaaactgagtggcctgaagaagccccccacgcttcagcccagcaaggaagcctgtttcctggagcatttgcacaaacaccagggctccgtcctgcaccccgactacaagacggccttcccctcctttgaggacgccctgcatcgcctcctgccctaccatgtctaccagggcgccctcccctcccccagtgactaccacaaagtggacgaggagtttgagacggtctccacgcagctgctgaaacgcacccaggccatgctcaataaatatcggctcctgctcctggaggagtcccggagggtgagcccctcagcggagatggtaatgatcgaccgaatgttcattcaggaggagaagaccacccttgccttggataaacagctggccaaggagaagccggacgagtacgtgtcttcctcccgctcgctcggcctccccatcgcagcctcttccgagggtcatcggcttcccggccacggccccctgtcgtcttcagctcccggggcctccacccagccccctccacacctgcccaccaagcttgtgatccggcacggcggggcaggcggctccccttcggtcacctgggcccgggcgtcctcctccctgtcctcctcttcctcctcctcctctgccgcctcctccttggacgccgacgaggacggccccatgccctcccgcaaccgcccgcccatcaagacctacgaggcccggagccgcatcgggctcaagctcaagatcaagcaggaagccgggctcagcaaggtcgtgcacaacacggccctggaccccgtgcaccagcccccgccaccccccgctaccctcaaggtggccgagcccccgccacggccgccaccaccaccgccgcccacgggccagatgaacggcacggtggaccacccgccgcctgccgcccccgagcgcaagcccctgggcaccgccccgcactgcccgcgcctgccactgcgcaagacctaccgcgagaacgtggggggccctggcgcgccggaggggacgcccgcaggcagggcacggggaggcagcccggcgccgctgcccgccaaagtggacgaggccaccagcgggctcatccgcgagctggcggccgtggaggacgagctgtaccagcgtatgctgaagggccccccgccagagcccgcagccagcgccgcccaaggcaccggggaccccgactgggaggcgcccgggctgccccctgccaagcggcgcaagtccgagtcgcccgacgtggaccaggccagcttctccagcgacagcccgcaggatgacacgctcaccgagcacctgcagagcgccatcgacagcatcctgaacctgcagcaggcccccggccggacgcccgcgccctcgtacccccacgctgcctcggccggcacccccgcatccccgccgcccctgcacaggcccgaggcctacccaccctccagtcacaacggtggcctcggcgccaggacgttgaccagataacaccgggccgcctccccttccccgtcccctcctcccgaagacgccgggacagtcgggtgtccgccctcagcctcctggggactcgagccggggatcccctgacggtttttcttgcctaagttatttgagtcacaaaggcctccttccctgccgcctgcttcagctgggttgctggggggtgggcgtggatttagggagggggctgtgatgtaaaacgtctcccctgccaaaggaggggcaaagtgctgtgtcagttcctgtttcttcccatttcctggcacactctgcccctctgtccgggggacacgcgcatgtgtttgccagggatggggccaccgggttgatgccaacgctccgggtgcctgtcttgtctgtgtggcttctcagatggtggagggtgctgggagctggcagggtccttccagacagtctcagcctctccccgccgcccccaacaggctgtcaaacaaaaccggagagggggtgggggagccagcctcccagcgtgctgtgcccgcaggcacccgtgtgacatccgcacgtccagctccgtgacctgtgtgtgtgtgtgtgcacaagtgagtgagagatttcgaacgcccacccctcgactttgaaatctgagcaaaacaagaaactggggtcttcctctcccccgaacctctccccagctagtcttccctctgttcttcctgcctccagccgcccgcgccagattttgaaatctcggagacaaaactagtactgtaagataaatttttttgtactgtatttattgtgtataacgatttttttaaaggagaat

SEQ ID NO: 3 - DNA, 282 bp, Homo sapiens
ggtcaggactcacttagtcttggttgatatcatcattatcatcatcaggtgctgggagagttggagtgggaggaaaaggaccctgtccctggtcaggggtttctggctgggagggcacctgggaggtattccttgtccccatctatccccatggactcaagggaacagaaactctgaccccctccagggggctcccctgaagggagggggccaggcaagctgccaaaatctaaccttgtctccctcgccctcttcctgaccccaccccatccagtggcgatg

SEQ ID NO: 4 - DNA, 390 bp, Homo sapiens
caccgaggctggagtgtagtgaagtgctcgtagctccactctgcagcctcaaactcctgggctccagtgatcctctcacctcagcctcccaagtagctaagaccacaggcacatgctgccacccctgactaatttttcttttttaaaatttttttctcactgcgttgcccaggctggtctcaaactctttggctcaagccatccatcctcctacctcagcctcccaaagtgctgagattacaagcgtgaaccaccatgccaggagggaatgttctttcttgaacgtggctgccctgtcacctcagcattctttgcggcagctgtggcctaagcgtagccccttgctaacctcagctctttccttcccacctttcccggcctgcagctcca

SEQ ID NO: 5 - DNA, 240 bp, Homo sapiens
ggtaagcagggcggggcaagggagcaggtaccggaggaggcgggtttgggaggaagaggggtcctggggcgaggcgccaagatccaaaagtggatgccactgtggcagctggggggggaaggtctcagtcatggcacaggtggcccctaagaggaggcccgaagggtgagggccctggctgaactggcaggaagatggattggcaggttggctgactgatagacaaggggcaggacaact

SEQ ID NO: 6 - DNA, 390 bp, Homo sapiens
cccccaggtagagggaccccagcagcctgtgtccgcagcacagacccagcctcgcccctcctgcccgctctcccgctcctcgctctccgccctgcctgcgtctctgttctgctgacttggaacacagagctctccttcaccccccaagtgtctcctagtgatcatggatgcctgagaccagggccggccctgggggagcccacgcgctgcaggaatgcaagacacacacggaaggaaatctgttctttcctaaaattataattttcaaatctgaataagtaatacattcagataattcacatttcaaacggtacaaaaagatgcatagtggaaagcccccgtctcctgaaccgtggcctcctgttgtcctcccaaggtggtttcttgcat

SEQ ID NO: 7 - DNA, 240 bp, Homo sapiens
ggggctgagcagggagaatgggctggggcagggctgtgggactcagcacagggcatcagccagaactcaagggacacaggaaggccagaggtggcagggctggccccagatttgaagggccggagggccaggaggagagcggtggggtacaggatggggctcccgtttgaggagggggccttcatgcagtgaccgcaggccctgtctcccccaccccacccacccccaggcctccctcct

SEQ ID NO: 8 - DNA, 300 bp, Homo sapiens
gccgcttcctccgggccagggaagccctccgggctgcaggtaaggggcccttagagcaggggtcaggacaggaccaggagtcttcgggaactgggaactggggctggcaggggcagggagacactggaagacgcggacaggatgaagctgggaccatcaggcagctgagacaccacgaacagccgtgcagggacaggaagggcagaatgacacccagacaggcagccagagatgagggagaaacacggagacggtgggacggagacggcaccaagactactggcaacagtgtaagggact

SEQ ID NO: 9 - DNA, 240 bp, Homo sapiens
ccatgtctaccagggcgccctcccctcccccagtgactaccacaaaggtgaggcctccccaggacacggccctatatgtcccaggggaccccagcccgtggggcggggcgtcgccagtgtggagccgcaggtccacggtgcgctatgctgaccctgccccgccctccttcctgcgcatccgcggccgcccccaacatctccgcccttgcctctcttcccttcctcgcagtggacgaggag

SEQ ID NO: 10 - DNA, 240 bp, Homo sapiens
tcctcgctgggacactgcccctttccctcacccgctctgggcaaggtggagcctcccgcccctcctagccccgggagggaggttgggagggaggcgggagctcccatcacaaggacagtttggaccttgcacgcatcgtccccgtcgctcgcgccccttcccccttcttgctggttcactcgcacgtcgtcttttcccccaccccagagggtgagcccctcagcggagatggtaatgatc

SEQ ID NO: 11 - DNA, 22 bp, Artificial Sequence
  Primer
ggcatggtcaggactcacttag

SEQ ID NO: 12 - DNA, 19 bp, Artificial Sequence
  Primer
agtgcctggcgttagaagc

SEQ ID NO: 13 - DNA, 20 bp, Artificial Sequence
  Primer
attcgaggcagtcacagcag

SEQ ID NO: 14 - DNA, 20 bp, Artificial Sequence
  Primer
ccaacagctcaggcaatgat

SEQ ID NO: 15 - DNA, 25 bp, Artificial Sequence
  Primer
agtgctaagattacaggcgtgagtc

SEQ ID NO: 16 - DNA, 21 bp, Artificial Sequence
  Primer
tgaggtaggaggatggatggc

SEQ ID NO: 17 - DNA, 20 bp, Artificial Sequence
  Primer
ctaagaccacaggcacatgc

SEQ ID NO: 18 - DNA, 25 bp, Artificial Sequence
  Primer
ttgggctctgggttcaggtggttgc

SEQ ID NO: 19 - DNA, 18 bp, Artificial Sequence
  Primer
ttccggcaaccacctgaa

SEQ ID NO: 20 - DNA, 19 bp, Artificial Sequence
  Primer
gccttgttgaccacgtcct

SEQ ID NO: 21 - DNA, 19 bp, Artificial Sequence
  Primer
caacatcacggagcagacg

SEQ ID NO: 22 - DNA, 22 bp, Artificial Sequence
  Primer
ccgagaggttcttctggatgac

SEQ ID NO: 23 - DNA, 20 bp, Artificial Sequence
  Primer
tcatccagaagaacctctcg

SEQ ID NO: 24 - DNA, 20 bp, Artificial Sequence
  Primer
acacgggcatctggaagagc

SEQ ID NO: 25 - DNA, 20 bp, Artificial Sequence
  Primer
gtggacagctcatcgcgaac

SEQ ID NO: 26 - DNA, 20 bp, Artificial Sequence
  Primer
tgccatgactgagaccttcc

SEQ ID NO: 27 - DNA, 20 bp, Artificial Sequence
  Primer
aaccctgggagcgtcaatag

SEQ ID NO: 28 - DNA, 20 bp, Artificial Sequence
  Primer
gtcagcagaacagagacgca

SEQ ID NO: 29 - DNA, 25 bp, Artificial Sequence
  Primer
gtgtcttggggcaacaggagctatg

SEQ ID NO: 30 - DNA, 25 bp, Artificial Sequence
  Primer
tcccatcagactgagagggtgggat

SEQ ID NO: 31 - DNA, 23 bp, Artificial Sequence
  Primer
ttccagatggtgaccaccccctt

SEQ ID NO: 32 - DNA, 20 bp, Artificial Sequence
  Primer
tgtgtcccttgagttctggc

SEQ ID NO: 33 - DNA, 22 bp, Artificial Sequence
  Primer
aactcaagggacacaggaaggc

SEQ ID NO: 34 - DNA, 23 bp, Artificial Sequence
  Primer
gtgtttctccctcatctctggct

SEQ ID NO: 35 - DNA, 22 bp, Artificial Sequence
  Primer
tatcctaggctagaggggaagc

SEQ ID NO: 36 - DNA, 20 bp, Artificial Sequence
  Primer
ctatgagggaactacatcgc

SEQ ID NO: 37 - DNA, 20 bp, Artificial Sequence
  Primer
ctcagtgagatggaacaagc

SEQ ID NO: 38 - DNA, 20 bp, Artificial Sequence
  Primer
tcaaactcctcgtccactgc

SEQ ID NO: 39 - DNA, 19 bp, Artificial Sequence
  Primer
caggtccacggtgcgctat

SEQ ID NO: 40 - DNA, 20 bp, Artificial Sequence
  Primer
cgagtgaaccagcaagaagg

SEQ ID NO: 41 - DNA, 20 bp, Artificial Sequence
  Primer
tcacaaggacagtttggacc

SEQ ID NO: 42 - DNA, 20 bp, Artificial Sequence
  Primer
cgccctgaatctaactcttc

SEQ ID NO: 43 - DNA, 18 bp, Artificial Sequence
  Primer
tccccatcgcagcctctt

SEQ ID NO: 44 - DNA, 19 bp, Artificial Sequence
  Primer
acgttctcgcggtaggtct

SEQ ID NO: 45 - DNA, 19 bp, Artificial Sequence
  Primer
agatgaacggcacggtgga

SEQ ID NO: 46 - DNA, 20 bp, Artificial Sequence
  Primer
gttcaggatgctgtcgatgg

SEQ ID NO: 47 - DNA, 20 bp, Artificial Sequence
  Primer
cgagctgtaccagcgtatgc

SEQ ID NO: 48 - DNA, 18 bp, Artificial Sequence
  Primer
gtccccaggaggctgagg

SEQ ID NO: 49 - DNA, 20 bp, Artificial Sequence
  Primer
aggtagtaggtgctcactgc

SEQ ID NO: 50 - DNA, 20 bp, Artificial Sequence
  Primer
tgccggatcacaagcttggt

(19) United States
(12) Patent Application Publication
(10) Pub. No.: US 2005/0255507 A1
(43) Pub. Date: Nov. 17, 2005

(54) Cytogenetically determined diagnosis and prognosis of proliferative disorders

(76) Inventors: Robert B. Jenkins, Rochester, MN (US); Ping Yang, Rochester, MN (US); Steve Thibodeau, Rochester, MN (US); Liang Wang, Rochester, MN (US); Daniel Schaid, Rochester, MN (US)

Correspondence Address: FISH & RICHARDSON PC, P.O. BOX 1022, MINNEAPOLIS, MN, 55440-1022, US

(21) Appl. No.: 11/059,982

(22) Filed: Feb. 17, 2005

Related U.S. Application Data
(60) Provisional application No. 60/545,573, filed on Feb. 17, 2004.

(51) Int. Cl. C12Q 1/68

(52) U.S. Cl. 435/6

(57) ABSTRACT

The invention is based, at least in part, on the discovery that mutations on the chromosome 19q arm can lead to a diagnosis that a human has an elevated likelihood of developing proliferative disorder, such as an oligodendroglioma or prostate cancer, and can further provide a prognosis of a human who has a proliferative disorder. A mutation can include a deletion of at least a fragment of a chromosome 19q arm, particularly at band 13.3, or an insertion or substitution of one or more nucleic acids on the chromosome 19q arm. Deletions and single nucleotide polymorphisms (SNPs) in the GLTSCR1 gene was found to be of particular predictive and diagnostic value.

### RELATED APPLICATIONS

This application claims the benefit of U.S. Provisional Application No. 60/545,573, filed Feb. 17, 2004, which is incorporated herein by reference in its entirety.

### GOVERNMENT SUPPORT

The work described herein was carried out, at least in part, using funds from the U.S. government under grant numbers CA85799, CA72818, and CA91956 awarded by the National Institutes of Health. The government may therefore have certain rights in the invention.

### TECHNICAL FIELD

This invention relates to predicting an occurrence of and determining prognoses for proliferative disorders, such as oligodendroglioma and prostate cancer, by assaying for genetic polymorphisms.

### BACKGROUND

Malignant gliomas are the most common primary central nervous system tumors affecting adults. While collectively referred to as diffuse gliomas, these tumors consist of a heterogeneous collection of tumor subtypes, including, among others, astrocytomas, oligodendrogliomas, and mixed oligoastrocytomas (MOAs). These glioma subtypes differ in their histologic appearance, as well as in their clinical presentation (including response to therapy, time to recurrence, and mortality). Previous molecular analyses have also demonstrated different genetic alterations associated with these subtypes. Tumors of astrocytic lineage often demonstrate anomalies of chromosome arms 9p, 10p, 10q, 11p, 13q, 17p, 17q, 19q, and 22q, whereas oligodendrogliomas and MOAs commonly have alterations of 1p and 19q. Oligodendrogliomas with 1p and 19q alterations have been observed to have a better survival and a better response to chemo- and/or radiation-therapy. Chromosome 19 q-arm alterations are the only known genetic abnormalities shared by all three glioma subtypes (Reifenberger et al., Am. J. Pathol. 145:1175-1538, 1994; von Deimling et al., Cancer Res. 52:4277-4279, 1992; Kraus et al., J. Neuropathol. Exp. Neurol. 54:91-95, 1995).

### SUMMARY

It has been discovered that mutations of the chromosome 19 q-arm (e.g., chromosome band 19q13.3) can indicate that a human has an elevated risk of developing a proliferative disorder, such as an oligodendroglioma or a cancer of the prostate, and that such mutations can further indicate a prognosis for a human who has a proliferative disorder. A mutation can include a deletion of at least a fragment of a chromosome 19q arm, such as at a particular band of 19q, including but not limited to band 13.3, or an insertion or substitution of one or more nucleotides on 19q. A mutation can also include a deletion or an insertion or substitution of one or more nucleotides in a gene located on 19q, such as in the GLTSCR1 gene. In particular, a single nucleotide polymorphism (SNP) in the gene GLTSCR1 was found to be of particular predictive and diagnostic value with respect to oligodendroglioma and prostate cancer. A human who has a T (thymine) at the position of the GLTSCR1 gene corresponding to position 1538 of SEQ ID NO:2 (see FIG. 2) can be diagnosed as being more likely to develop an oligodendroglioma and a prostate cancer than a human who has a C (cytosine) at that nucleotide position. The human can be homozygous, hemizygous, or heterozygous for T at the position. Further, a human who is homozygous for a T at the position, or who is hemizygous, or who has an oligodendroglioma that presents a GLTSCR1 gene that is hemizygous for a T at the position, has a better prognosis (a “good” prognosis, as described herein) than a human who has an oligodendroglioma and who is homozygous or heterozygous for a C at the nucleotide position. A human who is heterozygous or homozygous for a C at the nucleotide position has a “baseline” prognosis, which defines the survival time and/or response to treatment (e.g., radiation and/or chemotherapy) of a typical human who has an oligodendroglioma. A human who is heterozygous T at the nucleotide position can have a better prognosis than a human who is homozygous C at the position.

Described herein are methods for diagnosing a patient's risk for developing a proliferative disorder, such as an oligodendroglioma or a cancer of the prostate. According to one method, (i) a biological sample from a human (e.g., a human patient) is provided; (ii) a genetic analysis of the chromosome 19q arm from the biological sample is performed; and (iii) if a chromosomal abnormality is detected on 19q, the patient is determined to have an elevated risk for developing an oligodendroglioma or a cancer of the prostate. In particular, the genetic analysis can be of a GLTSCR1 nucleic acid (e.g., a GLTSCR1 DNA or RNA). The biological sample for performing the diagnostic and prognostic methods described herein can be, for example, a blood, saliva, urine, or tissue sample, including, but not limited to, a tumor sample or an epidermal tissue sample, such as a sample scraped from the inside of the cheek. A tumor sample can be, for example, from a glioma or a tumor of the prostate, e.g., from a tissue biopsy. Two samples from a patient can be tested for the presence of a chromosome 19q arm abnormality (e.g., a SNP), such as one sample from a tumor (to determine the tumor genotype), and one from an unaffected part of the body, such as from a cheek swab, to test for germline polymorphisms.

A genetic analysis can be, for example, a deletion mapping study or SNP analysis. Various methods for performing genetic analyses are known in the art and include, but are not limited to, FISH, homozygosity mapping, cytogenetics, spectral karyotyping (SKY), or comparative genomic hybridization (CGH) to arrays (e.g., microarray analysis or Affymetrix or other Gene chip-based methods).

A genetic analysis can be performed, for example, to detect a chromosomal abnormality on any chromosome, and particularly on a chromosome 19q arm (e.g., on 19q13.3). The genetic analysis can detect a deletion (e.g., a nucleotide deletion or chromosome deletion) or a nucleotide insertion or substitution. For example, a genetic analysis that detects a chromosome deletion can detect a chromosome 19q deletion (among other abnormalities), such as a chromosome 19q13.3 deletion, including a deletion of at least a fragment of the GLTSCR1 gene. For example, the genetic analysis can detect a deletion that includes at least a fragment of an exon of GLTSCR1, such as exon 6.

In addition, or in an alternative, a genetic analysis can detect an abnormality on a chromosome 19q arm that includes a SNP in the GLTSCR1 gene, such as in exon 6 of GLTSCR1 (see Table 11). For example, a SNP in exon 6 of GLTSCR1 can be a C to T substitution in the codon encoding the amino acid of a GLTSCR1 polypeptide corresponding to amino acid 448 of SEQ ID NO:1 (see FIG. 1). The substitution can be at the nucleotide position of a GLTSCR1 nucleic acid corresponding to position 1538 of SEQ ID NO:2. The SNP can be any of those listed in Tables 7, 10, and 11.

A genetic analysis of a biological sample from a human may reveal that the human (e.g., patient) is homozygous for a C at the position corresponding to position 1538 of SEQ ID NO:2, in which case it can be determined that the human (e.g., patient) does not have an elevated risk of developing an oligodendroglioma. In fact, the human can be determined to have a relatively low risk of developing an oligodendroglioma.

Optionally, the diagnostic and prognostic methods described herein can include notifying the human or a caregiver of the human of any diagnoses and/or prognoses resulting from the methods, and further the diagnoses and prognoses can be recorded, such as in print or in a computer-readable format. A “caregiver” can be any entity involved with providing care to the human: for example, a hospital, hospice, doctor's office, outpatient clinic; a healthcare worker such as a doctor, nurse or other practitioner; or a spouse or guardian, such as a parent.

Methods for determining a prognosis for a glioma patient (e.g., an oligodendroglioma patient) are also described herein. In one method, (i) a biological sample (e.g., a glioma sample) is provided from a patient; (ii) a SNP analysis is performed (e.g., on the section of a chromosome 19q arm that includes the GLTSCR1 gene, and in particular, the nucleotide position in the GLTSCR1 gene corresponding to position 1538 of SEQ ID NO:2) on the glioma sample; and (iii) the patient is determined to have (A) a good prognosis if the patient is homozygous or hemizygous for a T at the nucleotide position corresponding to position 1538 of SEQ ID NO:2, or (B) a baseline prognosis if the patient is heterozygous, hemizygous, or homozygous for a C at the position corresponding to position 1538 of SEQ ID NO:2.

Methods for diagnosing a patient as having an elevated risk of developing prostate cancer are also provided. Such methods include providing a biological sample from a patient, performing a genetic analysis of the chromosome 19q arm from the biological sample, and detecting a SNP at one or more of the nucleotide positions reported in Tables 7, 10, and 11. The presence of a SNP indicates that the patient has an elevated risk of developing prostate cancer. More particularly a SNP located at one or more of positions 1344, 1538, 1768, 2241, 2668, 2781, 3324, or 4618, as defined by SEQ ID NO:2, can indicate that the patient has an elevated risk of developing prostate cancer.

Also provided are kits including reagents and informational material, such as instructions, for determining the genotype of a human of the chromosome 19q arm, and determining the human's risk of developing a proliferative disorder, such as an oligodendroglioma or prostate cancer. Kits are also provided for determining a human's prognosis for surviving a proliferative disorder, such as an oligodendroglioma. A kit can include reagents for FISH or comparative genomic hybridization to arrays, or any method of genetic analysis described herein.

Unless otherwise defined, all technical and scientific terms used herein have the same meaning as commonly understood by one of ordinary skill in the art to which this invention pertains. The materials, methods, and examples are illustrative only and not intended to be limiting. Although methods and materials similar or equivalent to those described herein can be used in the practice or testing of the present invention, useful methods and materials are described below. Other features and advantages of the invention will be apparent from the accompanying drawings and description, and from the claims. The contents of all references, pending patent applications and published patents, cited throughout this application are hereby expressly incorporated by reference. In case of conflict, the present specification, including definitions, will control.

### DESCRIPTION OF DRAWINGS

FIG. 1 is the amino acid sequence of GLTSCR1.

FIG. 2 is the mRNA sequence of GLTSCR1. The ATG start codon is marked in bold.

FIG. 3 is a map summarizing the 19q deletions in cell lines A172, U87 and primary gliomas. Chromosome region 19q13.2-q13.42 is enlarged to show base position (in Mbs) and BAC contig based on Lawrence Livermore National Laboratory chromosome 19 map. The block bar on the right indicates the minimal 19q deletion region mapped in gliomas (Smith et al., Genes Chromosomes Cancer 29:16-25, 2000). The markers D19S902 and D19S246 are highlighted because they have been previously associated with aggressive prostate carcinoma and adenocarcinoma of the lungs, respectively (Slager et al., Am J Hum Genet 72:759-762, 2003; Yanagitani et al., Cancer Epidemiol. Biomarkers Prevent. 12:366-371, 2003).

FIG. 4 a gene map of the A172, U87 and primary glioma 19q13.3 deletion region. The map includes genes, expressed sequence tags (ESTs), or computationally identified open reading frames (ORFs) believed to have putative cancer-related function or to have homology to genes with putative cancer-related function, genes previously used in epidemiologic studies, or genes, ESTs or computationally identified ORFs underexpressed in oligodendrogliomas with 19q deletion (the latter are indicated by arrows). The map also includes genes containing the SNPs that were evaluated in the examples described below.

FIG. 5 is a sequence fragment of the 3′ end of intron 2. Small-case letters represent intron 2 sequence; capital letters represent exon 3 sequence. The ATG in exon 3 is the translation start codon of GLTSCR1. The sequence is from nucleotide “1-250” through nucleotide “3” according to the numbering scheme of Table 11.

FIG. 6 is a sequence fragment of the 3′ end of intron 5. Small-case letters represent intron 5 sequence; capital letters represent exon 6 sequence. The sequence is from nucleotide “151-385” through nucleotide “155” according to the numbering scheme of Table 11.

FIG. 7 is a sequence fragment of the 5′ end of intron 6. Small-case letters represent intron 6 sequence; capital letters represent exon 5 sequence. The sequence is from nucleotide “2106” through nucleotide “2106+239” according to the numbering scheme of Table 11.

FIG. 8 is a sequence fragment of the 5′ end of intron 7. Small-case letters represent intron 7 sequence; capital letters represent exon 6 sequence. The sequence is from nucleotide “2277” through nucleotide “2283+383” according to the numbering scheme of Table 11.

FIG. 9 is a sequence fragment of the 3′ end of intron 9. Small-case letters represent intron 9 sequence; capital letters represent exon 10 sequence. The sequence is from nucleotide “3077-229” through nucleotide “3087” according to the numbering scheme of Table 11.

FIG. 10 is a sequence fragment of the 5′ end of intron 10. Small-case letters represent intron 10 sequence; capital letters represent exon 9 sequence. The sequence is from nucleotide “3148” through nucleotide “3186+261” according to the numbering scheme of Table 11.

FIG. 11 is a sequence fragment including intron 12 and the ends of the flanking exons. Small-case letters represent intron 12 sequence; capital letters represent exon sequences.

FIG. 12 is a sequence fragment of the 3′ end of intron 13. Small-case letters represent intron 13 sequence; capital letters represent exon 14 sequence. The sequence is from nucleotide “3493-207” through nucleotide “3525” according to the numbering scheme of Table 11.

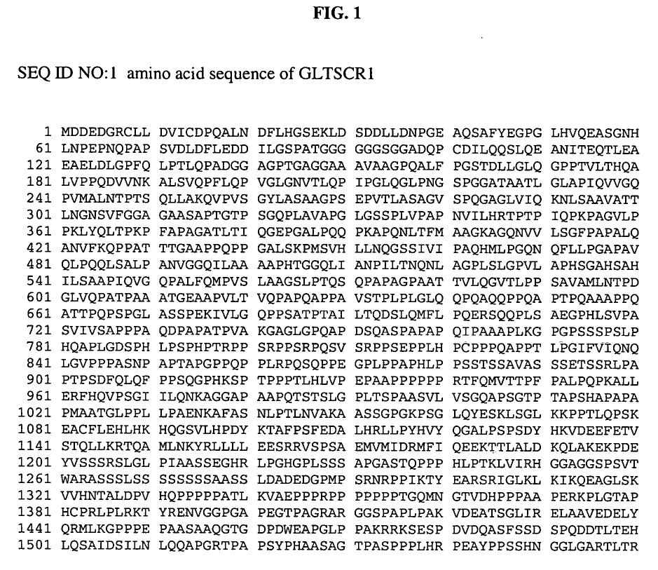

### DETAILED DESCRIPTION

The methods described herein can be used to diagnosis a proliferative disorder, such as an oligodendroglioma or a cancer of the prostate, or to determine a prognosis for the development and/or survival of a proliferative disorder.

A “proliferative disorder” is a disorder characterized by irregularities in cell division. A cancer (e.g., a glioma, prostate cancer, melanoma, carcinoma, cervical cancer, breast cancer, colon cancer, or sarcoma) is an example of a proliferative disorder. Cells characteristic of proliferative disorders, including tumor cells, have the capacity for autonomous growth, i.e., an abnormal state or condition characterized by inappropriate proliferative growth of cell populations. Proliferative disorders include all types of cancerous growths or oncogenic processes, metastatic tissues or malignantly transformed cells, tissues, or organs, irrespective of histopathologic type or stage of invasiveness. Cancers include malignancies of various organ systems, such as the lung, breast, thyroid, lymphoid, gastrointestinal, and genito-urinary tract, as well as adenocarcinomas, which include malignancies such as most colon cancers, renal-cell carcinoma, prostate cancer and/or testicular tumors, non-small cell carcinoma of the lung, cancer of the small intestine and cancer of the esophagus. Carcinomas include malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Other carcinomas include those forming from tissue of the cervix, lung, head and neck, colon and ovary. Cancers of the central nervous system include gliomas, (including astrocytomas, mixed oligoastrocytomas, glioblastoma multiform, ependymoma, and oligodendroglioma), meningiomas, pituitary tumors, hemangioblastomas, acoustic neuromas, pineal gland tumors, spinal cord tumors, and lymphomas.

An oligodendroglioma is a type of glioma, named after the cells from which it originates, oligodendrocytes. Typically, such tumors have an indolent course, and patients can survive for many years after symptom onset. Oligodendrogliomas arise in the cerebral hemispheres and are classified as low grade or anaplastic. They usually occur in the cerebral white matter and are very cellular, with uniform nuclei. They also typically grow outward from white matter into gray matter and are relatively avascular.

An elevated incidence of oligodendrogliomas has been correlated with abnormalities (e.g., deletions, SNPs) on the chromosome 19q arm. Particularly, oligodendrogliomas have been correlated with 19q deletions, particularly at 19q13.3. Further, a SNP in a gene located in the 19q13.3 region has been correlated with an elevated incidence of oligodendroglioma. The gene, GLTSCR1, exhibits no homology with any known genes. The flanking and UTR regions are exceedingly GC-rich.

An elevated incidence of prostate cancer has also been correlated with abnormalities on the chromosome 19q arm. Particularly, the incidence of prostate cancer has been correlated with SNPs in the GLTSCR1 gene.

Diagnostic Methods A human determined to have an abnormality on the chromosome 19q arm can be determined to have an elevated risk of developing a proliferative disorder, such as an oligodendroglioma or a cancer of the prostate. The human may have an elevated risk of developing an oligodendroglioma and a cancer of the prostate, as well as other forms of cancer, including cancers of the colon, breast, lung, liver, uterus, cervix, and skin (e.g., a carcinoma, such as an adenocarcinoma, or a basal cell or squamous cell carcinoma). An “elevated risk” is a risk greater than that of a human who does not carry an abnormality on the chromosome 19q arm (e.g., the human does not carry a 19q deletion or SNP).

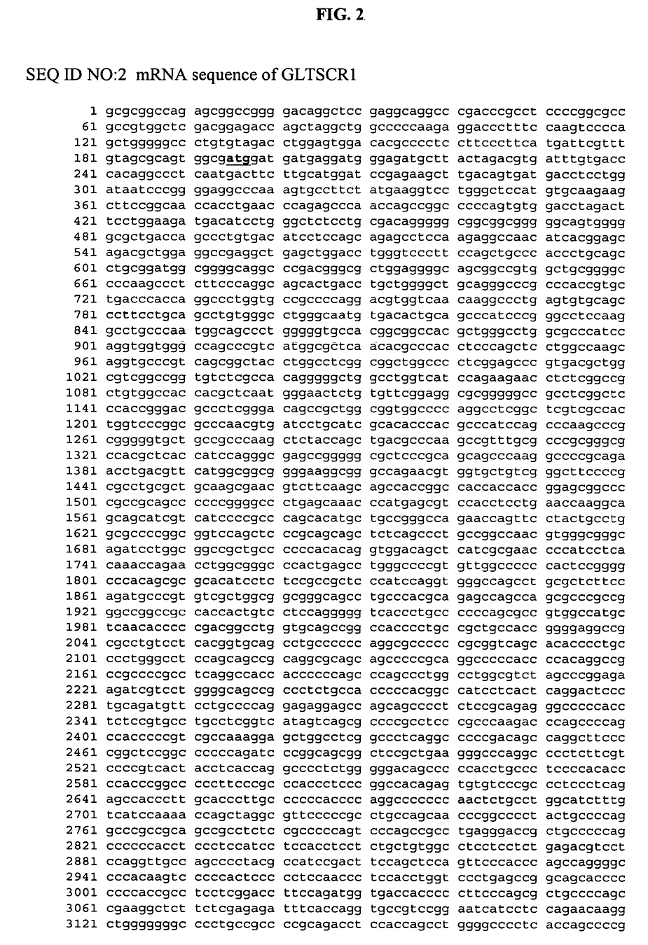

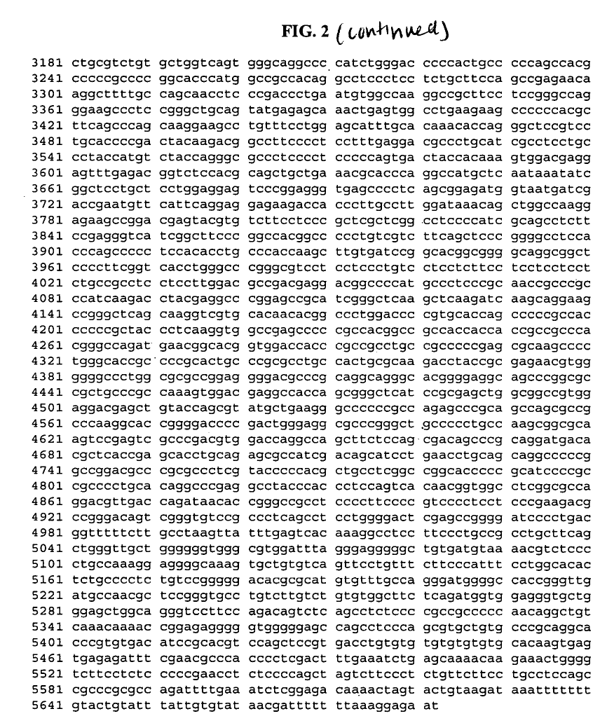

The genetic analysis methods described herein can identify chromosomal abnormalities including deletions, translocations and polymorphisms. The term polymorphism includes nucleotide substitution, nucleotide insertion and nucleotide deletion, which in the case of insertion and deletion, includes insertion or deletion of one or more nucleotides at a position of a gene. Polymorphisms also include SNPs (single nucleotide polymorphisms). A human diagnosed as having an elevated risk of developing an oligodendroglioma, for example, can have a chromosome 19q arm deletion, such as a 19q13.3 deletion, or a T within at least one allele of exon 6 of the GLTSCR1 gene at the position corresponding to position 1538 of SEQ ID NO:2 (see FIG. 2). A human who has an “elevated risk” for developing an oligodendroglioma has a higher risk than a human who is homozygous for a C at that nucleotide position. As described herein, reference to a T or a C on one allele (one strand of a DNA molecule) at the designated nucleotide position equates an A or G, respectively, at the same position on the complementary strand of DNA (see Examples 4-7 below).

Methods for diagnosing a human's risk for developing a proliferative disorder, such as an oligodendroglioma or prostate cancer, are described herein. For example, a genetic analysis can be performed to assay for a chromosomal abnormality, such as an abnormality on the chromosome 19q arm (e.g., a deletion or SNP). The analysis can be performed on a biological sample of a human. If an abnormality is a SNP in the GLTSCR1 gene, such as in exon 6 of GLTSCR1, the abnormality can indicate that the human has an elevated risk for developing an oligodendroglioma. For example, a SNP can be in exon 6 of GLTSCR1, and the SNP can be a C to T substitution at the position corresponding to position 1538 of SEQ ID NO:2 (see FIG. 2). The SNP can be in one or both alleles of GLTSCR1, and can lead to a diagnosis of the human as being at an elevated risk for developing an oligodendroglioma. A human determined to be homozygous for a T at the nucleotide position can be diagnosed as being at a greater risk for developing an oligodendroglioma than a human determined to be heterozygous for a T at the nucleotide position.

A genetic analysis of a biological sample from a human may reveal that the human (e.g., patient) is homozygous for a C at the position corresponding to position 1538 of SEQ ID NO:2, in which case the human can be diagnosed as not having an elevated risk of developing an oligodendroglioma; in fact, the human can be diagnosed as having a relatively low risk for developing an oligodendroglioma.

In another example, a SNP can indicate an elevated risk of developing prostate cancer. For example, a SNP can be any SNP listed in Tables 7, 10, and 11. In particular, the SNP can be a C to T substitution at the position corresponding to position 1538 of SEQ ID NO:2; a C to T substitution at position 2241 of SEQ ID NO:2; a C to G substitution at position 1344 of SEQ ID NO:2; a G to A substitution at position 1768 of SEQ ID NO:2; a C to T substitution at position 2668 of SEQ ID NO:2; a C to T substitution at position 2781 of SEQ ID NO:2; a A to G substitution at position 3324 of SEQ ID NO:2; or a G to A substitution at position 4618 of SEQ ID NO:2. Alternatively or in addition, the SNP can be a G to A substitution in GLTSCR1 intron 7 corresponding to the NCBI refSNP ID No. rs2914430, or a T to G substitution in the region 3′ of the GLTSCR1 coding region and corresponding to the NCBI refSNP ID No. rs1005911 (see Tables 9 and 10).

The diagnostic methods can be performed on any human at any age, including a fetus (e.g., in utero), infant, toddler, adolescent, adult, or elderly human.

Prognostic Methods A prognosis can be provided for a patient diagnosed with a proliferative disorder, such as a glioma patient (e.g., an oligodendroglioma patient). For example, a patient homozygous or hemizygous for a T at the position in exon 6 of the GLTSCR1 gene corresponding to position 1538 of SEQ ID NO:2 can be determined to have a good prognosis. A human with a good prognosis is likely to live longer than a human with a “baseline” prognosis. A human with a good prognosis is also likely to recover fully or partially, or at least respond favorably to treatment regimens, including chemotherapy, radiation therapy, and other treatment regimens undertaken to reduce or eliminate a glioma. A patient heterozygous, hemizygous, or homozygous for a C at the position of exon 6 of the GLTSCR1 gene corresponding to position 1538 of SEQ ID NO:2 can receive a “baseline” prognosis. A baseline prognosis is a measure of survival time or response to therapy to which a human being hemizygous or homozygous for a T at the nucleotide position is compared. A human with a baseline prognosis is not likely to survive for as long a period of time as a person with a good prognosis, and a human with a baseline prognosis may not respond as well to treatment with chemotherapy, radiation therapy, and other treatment regimens undertaken to reduce or eliminate a glioma as a person with a good prognosis.

Data obtained from the methods featured herein can be combined with information from a patient's medical records, including demographic data; vital status; education; history of alcohol, tobacco and drug abuse; medical history; and documented treatment to adjust conclusions relating to diagnosis and prognosis of a proliferative disorder.

DNA Analysis Methods Detection of chromosomal abnormalities, including deletions and SNPs, can be identified by methods known in the art, including fluorescent in situ hybridization (FISH), comparative genomic hybridization (CGH), homozygosity mapping, cytogenetics, spectral karyotyping (SKY), Southern and Northern blot analysis, PCR (including allele-specific PCR extension and amplification protocols), reverse transcription-coupled polymerase chain reaction (RT-PCR), restriction fragment length polymorphism (RFLP) analysis, Taqman™, Molecular Beacons, restriction based PCR, fluorescence resonance energy transfer (FRET) techniques, or direct sequencing. Array-based methods employing such methods can also be used. For example, allele-specific PCR extension and amplification can be coupled with an array-based optical detection method to analyze large numbers of SNPs. Packaged systems such as Pyrosequencing™ (Biotage, Charlottsville, Va.), ABI SNP-plex™ (Applied Biosystems, Foster City, Calif.) and Affymetrix SNP-chip™ (Affymetrix, Santa Clara, Calif.) analysis can be performed to manage high-throughput analysis of biological samples for SNP identification.

Kits Reagents, tools, and instructions for performing the methods described herein can be provided in a kit. The informational material can be descriptive, instructional, marketing or other material that relates to the methods described herein and/or the use of the reagents for the methods described herein. For example, the informational material can relate to performing a genetic analysis on a human and subsequently diagnosing the human as being at risk (or not) for a proliferative disorder, such as an oligodendroglioma or prostate cancer, and/or delivering a prognosis of the human relating to survival time, likelihood of responding to therapy, etc. In addition, or in an alternative, the informational material of the kit can be contact information, e.g., a physical address, email address, website, or telephone number, where a user of the kit can obtain substantive information about performing a genetic analysis and interpreting the results, particularly as they apply to a human's likelihood of developing a proliferative disorder (e.g., an oligodendroglioma) and a subsequent prognosis.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. Of course, the informational material can also be provided in any combination of formats.

The kit can include one or more containers for the reagents for performing a genetic analysis, such as reagents for performing FISH, CGH, or any other technique described herein. The kit can contain separate containers, dividers or compartments for the reagents and informational material. A container can be labeled for use for the diagnosis and/or prognosis of a human relating to the development and treatment of a proliferative disorder.

The invention is further illustrated by the following examples, which should not be construed as further limiting.

### EXAMPLES

### Example 1

### Identification of Test Subjects and Control Subjects, and Collection of Biological Samples

We have described a transcript map of the minimally-deleted 19q region in gliomas (Smith et al., Genomics 64:44-55, 2000). Three novel transcripts (EHD2, GLTSCR1 and GLTSCR2) and two known genes (SEPW1 and CRX) map to this deletion region (GLTSCR=glioma tumor suppressor critical region).

A general control group was drawn from an existing and ongoing Lung Cancer Research Program (funded by R01-80127, R01-84354, and R03-87701 to Dr. Yang) at the Mayo Clinic Cancer Center (MCCC). These controls were residents of Olmsted County where Mayo Clinic (Rochester site) is located and serves as a major primary care facility. There were two purposes for this population-based control group. The first was to serve as a reference in testing our hypotheses; and the second was to provide an accurate estimate of the expected allele frequencies of the candidate SNPs in a reference population. Olmsted County residents, who had neither currently nor previously diagnosed glioma nor other invasive malignancies (except for non-melanoma skin cancer) or major organ failure as of the date of the blood draw at the Mayo Clinic, were eligible as controls. The advantage and the main reason for this design were time- and cost-effectiveness considering a reasonable response rate (given below). Justification of this design was based on findings from the Rochester Epidemiology Project (Melton, Mayo Clin. Proc. 71: 266-274, 1996), which showed that over a three-year period, over 90% of Olmsted County residents will visit the Mayo Clinic at least once with a blood draw. We utilized a centralized computer system, which tracked blood samples received by the Mayo Clinic Central Processing Unit from all outpatient and emergency room patients in Rochester. Only individuals granting a general research authorization for research use of their medical records were listed. This list was matched with the Mayo Clinic's patient registration databases, and a daily list produced containing the identification number of each sample from any Olmsted County resident.

An extra amount of blood was removed from each patient at the initial phlebotomy, and his or her blood samples were held in Mayo Clinic Central Processing (at 4° C.) for three days before being discarded (in case of a need for repeated or additional tests). After potential controls were identified, their samples were collected on the third day, plasma and Buffy coat separated, and placed in a −70° C. freezer for further processing. We wrote to these patients asking their permission to use their blood samples and invited them to participate in our study as community controls by filling out a study questionnaire, which contained identical questions and format as in the patient interview. Blood samples were either discarded or stored for research depending on the status of the informed consent. Two to six months were required to obtain a matched control for each enrolled case. A total of 1,655 controls (age range, 18 to 97 years) were enrolled and available to be matched to glioma cases.

A case-control study of ovarian cancer has been ongoing at MCCC (R01 CA86888), and has successfully implemented a protocol for identifying and recruiting control subjects from the departments of Internal Medicine at Mayo Clinic. Patients who were scheduled for regular general medical exams were sent letters 3 weeks prior to their appointment, informing them of their opportunity to serve as a healthy “control” for cancer research. A study coordinator met them at the time of their scheduled visit to discuss participation, obtain informed consent, and arrange the venipuncture (usually done at the same time as required for the general medical exam). The response rate was between 57% and 87%, with a conservatively estimated response rate of 70%. Ninety-four percent of consenting women gave a blood sample. The same model was implemented for enrolling men as controls in the MCCC.

### Example 2

### Chromosome 19 Deletion Mapping

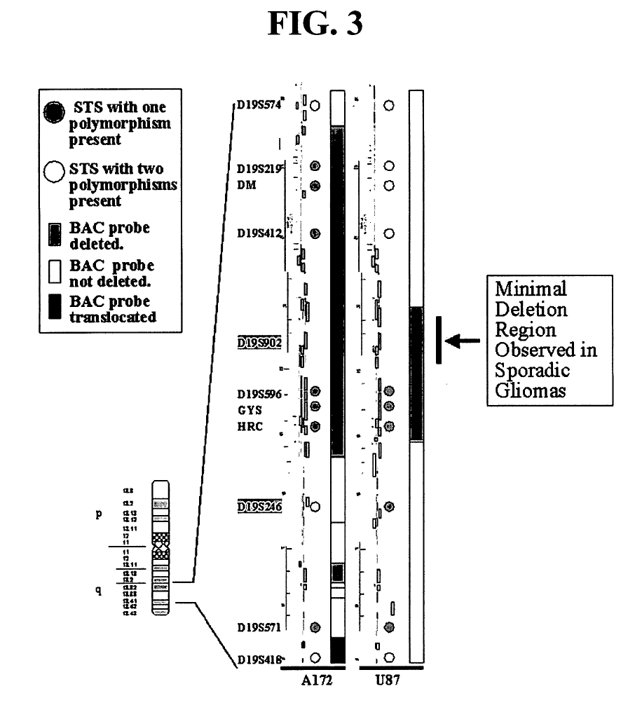

We have previously described a 150 kb minimally deleted 19q region in gliomas using combined FISH and LOH analyses (Smith et al., Genes Chromosomes Cancer 29:16-25, 2000). Since that publication we have evaluated the 19q deletion status of 17 glioma cell lines using FISH, homozygosity mapping, routine cytogenetics, spectral karyotyping and comparative genomic hybridization to arrays (CGHa). Two cell lines, U87 and A172, have 19q microdeletions that completely encompass the deletion region that we previously mapped in primary gliomas (FIG. 3). The A172 deletion is approximately 4.5 Mb in size.

### Example 3

### Genome Map of A172, U87 and Primary Gliomas 19q Deletion Regions

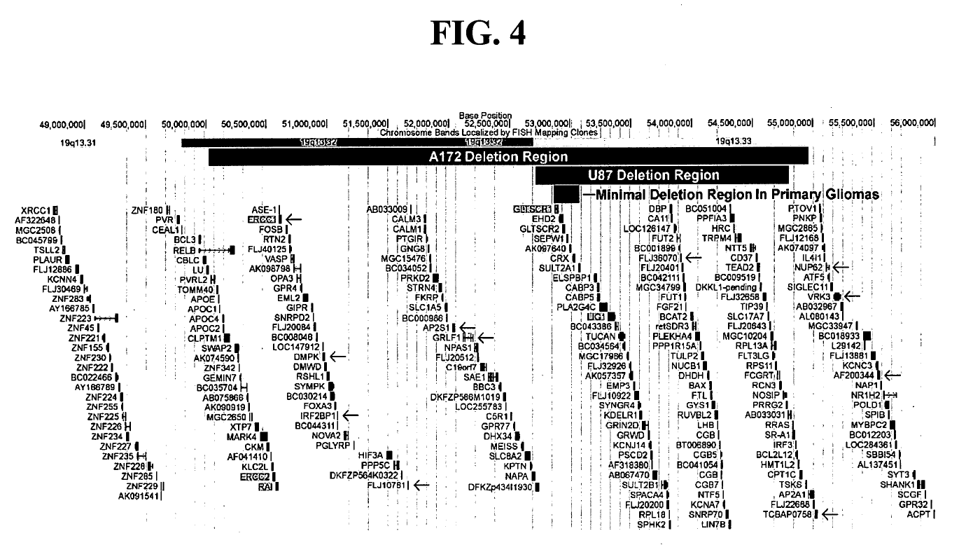

FIG. 4 summarizes the gene map of the glioma 19q deletion region (NCBI build 34). The boundaries of the illustrated map are limited by the deletion region defined by the A172 glioma cell line; the U87 and primary glioma deletions are also indicated. Both known genes and computationally identified genes are illustrated. There are 124 known genes and 49 computationally (or genes assembled from ESTs) identified genes in the A172 primary glioma deletion region. The NCBI SNP database lists 5718, 3046, and 251 SNPs for the A172, U87 and primary glioma deletion regions, respectively.

### Example 4

### Risk of Glioma Development

We carried out a pilot SNP association study, based on the information and blood specimens collected from 251 patients, 143 glioma cases and 108 general controls (the latter were Olmsted County residents). We sub-classified the 143 neuro-oncology patients according to their tumor morphology (Astrocytoma N=61, Oligodendroglioma N=42, Mixed N=40). Each of these 3 morphologic groups was then compared to the control group in the analysis. Univariate associations of allele (which treats each chromosome as a unit) and genotype (which treats a person as a unit) with disease were evaluated using contingency table methods in SAS v8.2. The multiple SNP marker-disease association with haplotype was evaluated using haploscore (a Mayo-developed package of S-plus functions), which accounts for ambiguous linkage phase (Schaid et al., Am. J. Hum. Genet. 70: 425-434, 2001). Linkage disequilibrium was assessed using the Graphical Overview of Linkage Disequilibrium (GOLD) software package (Abecasis and Cookson, Bioinformatics 16:182-183, 2000).

Table 2 summarizes the morphology and age distributions of the cases and the age distribution of controls. Table 3 summarizes the 7 SNPs in 5 genes that were analyzed. Note that the SNPs are located on the noncoding strand of the DNA, and thus, for example, the G to A polymorphisms of GLTSCR1 illustrated in Table 3 equate to the C to T polymorphism at the same position (the nucleotide position corresponding to position 1538 of SEQ ID NO:2) on the coding strand of DNA, as described herein.

These 7 SNPs had previously been shown to be associated with basal cell carcinomas, breast cancers or mixed oligoastrocytomas (Dybdahl et al., Cancer Epidemiol. Biomarkers Prevent. 8:77-81, 1999; Rockenbauer et al., Carcinogenesis 23:1149-1153, 2002; Yin et al., Cancer Epidemiol Biomarkers 11:1449-1453, 2002; Nexo et al., Carcinogenis 24:899-904, 2003; Chen et al., Cancer Epidemiol. Biomarkers Prevent. 9:843-847, 2000; Caggana et al., Cancer Epidemiol. Biomarkers Prevent. 10:355-360, 2001). They also map within the A172 deletion region. Pyrosequencing™ was used to determine SNP genotypes.

TABLE 1
Glioma and Blood Specimens Currently Available
(Accrued 1990-2003)

| | | Leukocyte or | EBV Line |
|---|---|---|---|
| | | Lymphoblast | (DNA potentially |
| | Tumor* | DNA prepared | available) |
| Mayo: Oligodendroglioma | 123 | 74 | 62 |
| Mixed Oligoastrocytoma | 109 | 51 | 62 |
| Grade 2-3 Astrocytoma | 39 | 16 | 4 |
| Glioblastoma | 211 | 89 | 27 |
| RTOG 9402**: | 204 | 0 | 264 |
| Anaplastic | | | |
| Oligodendroglioma | | | |
| or mixed oligoastrocytoma | | | |

*For Mayo Clinic patients frozen and paraffin-embedded tumor specimens are available. For RTOG 9402 only paraffin sections are available.
**We are currently blinded to the specific tumor morphologic diagnosis entered on RTOG 9402.

TABLE 2
Age Distribution of Cases and Controls
in Pilot Glioma SNP Association Study

| | Percentage of Individual | | |
|---|---|---|---|
| | by Age Group | | |
| | <51 Years | 51-60 Years | >60 Years |
| Control | 9 | 41 | 50 |
| Astrocytoma | 42 | 15 | 43 |
| Mixed Oligoastrocytoma | 72 | 15 | 13 |
| Oligodendroglioma | 88 | 12 | 0 |

TABLE 3
SNPs Analyzed in Pilot Glioma Association Study

| | | | Function | Alleles |
|---|---|---|---|---|
| Gene | Location | RS#* | of Change | (frequencies)** |
| ERCC1 | Exon1 | rs3212986 | Q504K | A(0.24), C(0.76) |
| ERCC1 | Exon 4 | rs3177700 | N118N | C(0.37), T(0.63) |
| ERCC2 | Exon 6 | rs338406 | R156R | G(0.53), T(0.47) |
| ERCC2 | Exon 22 | rs1052555 | E542E | A(0.31), G(0.69) |
| GLTSCR1 | Exon 6 | rs1035938 | S397S*** | A(0.25), G(0.75) |
| Lig1 | Exon 6 | rs20580 | A170A | A(0.48), C(0.52) |

*RS# = Accession number in NCBI SNP database
**frequencies found among general controls in a pilot study; nucleotides are from the non-coding DNA strand, and thus the G to A SNP of GLTSCR1 (bold) is equivalent to the C to A SNP described herein
***S397 corresponds to S448 of SEQ ID NO: 1

We compared allele frequencies of each of the 7 SNPs between general controls and glioma cases by morphologic subtypes (Table 4). The presence of a germline GLTSCR1-exon-6 A allele was significantly associated with the development of oligodendroglioma (p=0.016).

The association between ERCC2-exon-22 G allele achieved borderline significance (P=0.093). No other associations between SNP alleles and oligodendroglioma or other glioma types were observed. Very similar and statistically significant associations were observed when the data was analyzed using genotype-based and carrier-based methods. For example, combined AA and AG genotypes for the GLTSCR1 -exon-6 locus were observed in 60% of oligodendroglioma patients, in contrast to 41% in mixed oligoastrocytoma or 48% in astrocytoma patients (P=0.04). The unadjusted odds ratio (as a measure of relative risk) for the development of oligodendroglioma associated with GLTSCR1 locus was 3.3 (95% CI 1.0-10.4) and 2.1 (95% CI 1.2-4.5) for the AA and AA/AG genotypes compared to the GG genotype, respectively. Among GLTSCR1 genotypes AA, AG, and GG, we observed AA in 18% of oligodendroglioma patients but only in 5% each in mixed oligoastrocytoma or astrocytoma patients (P=0.02). Interestingly, of the 12 patients with the AA genotype, 7 (58%), 2 (18%) and 3 (25%) developed oligodendrogliomas, mixed oligoastrocytomas and astrocytomas, respectively.

While the controls were drawn from the Olmsted County normal control pool, they were not formally matched by age or gender to the glioma cases. The significance of the above associations did not change when the analyses were stratified by age (grouped as <50, 50-60 and >60) and gender.

As a demonstration, we also performed 2-locus analysis to detect potential gene-gene interactions. The risk for developing oligodendroglioma of individuals who were homozygous AA at GLTSCR1 locus remained the same (OR=3.3, 95% CI: 1.0 to 10.6) after adjusting for ERCC1-exon-1 genotype.

Table 5 summarizes the haplotype-based analysis of the patients with oligodendrogliomas. A total of 25 haplotypes were identified and only haplotypes of frequency 0.03 or higher in either cases or controls are shown. Our preliminary data showed that one high-risk (CCGAAAA) and one low-risk (ACGAGAA) haplotype were identified (simulated p=0.006 and 0.048, respectively). These two haplotypes differ only by the presence of an ERCC1-exon1 allele (C or A) or GLTSCR1-exon-6 allele (A or G). The high-risk haplotype was computationally identified by the DNA Markers program in three patients, and only in patients with oligodendroglioma, never in a patient with another glioma or in a control. When the haplotype-data was statistically examined as a whole, these two haplotypes are related to oligodendroglioma development (max-stat simulated p-value=0.024, 25 degrees of freedom). There were no haplotypes associated with astrocytoma and mixed oligoastrocytoma development in this pilot study.

### Example 5

### Correlations Between Germline SNPs with Glioma 19q Deletion Status

We carried out a stratified analysis to determine if there were associations between the various SNPs we evaluated and glioma 19q deletion status. The GLTSCR1-exon-6 A allele was associated with glioma 19q deletion status: 61% of patients whose glioma had 19q deletion carried the germline A allele versus 31% of those without deletion (p=0.05) (note that the “GLTSCR1-exon-6 A allele” refers to a SNP located on the noncoding strand of the DNA, and thus equates to a T nucleotide at the same position on the coding strand of DNA, i.e., a T at the nucleotide position corresponding to position 1538 of SEQ ID NO:2). The oligodendrogliomas carrying a 19q deletion had significantly higher frequency of the “A allele” (0.42) than the controls (0.24), whereas oligodendrogliomas without a 19q deletion demonstrated a significantly lower frequency of the “A allele” (0.17) as compared to controls (0.24) (p=0.02). Haplotype analysis was performed in 21 and 10 oligodendroglioma cases with and without a 19q deletion, respectively. Two new high-risk haplotypes were identified in the group with a 19q deletion: (CTGGAAT, p<0.05) and (CTGGACT, p<0.001). No haplotypes were identified in the group lacking a 19q deletion. The newly identified haplotypes are very similar to the high-risk haplotype identified for the oligodendrogliomas as a whole and share the ERCC1-exon-1 C and the GLTSCR1-exon-6 A alleles. Significant linkage disequilibrium (LD) was found between ERCC1-exon-6 and ERCC1-exon-4, and between ERCC2-exon-6 and RAI-exon-6. The number of mixed oligodendrogliomas and astrocytomas with 19q loss was too small for stratified analysis.

### Example 6

### Correlations with Tumor Genotype to Identify the Lost Alleles

Seventeen oligodendrogliomas were heterozygous for the GLTSCR1-exon-6 A and G alleles (note that the “GLTSCR1-exon-6 A and G alleles” refers to nucleotides located on the noncoding strand of the DNA, and thus equate to a T or C, respectively, at the same position on the coding strand of DNA, i.e., at the nucleotide position corresponding to position 1538 of SEQ ID NO:2). We evaluated 11 of these tumors to determine which of the two alleles were lost. Paraffin sections from all 11 tumors were evaluated by fluorescent in situ hybridization (FISH) using a BAC probe (labeled with a red fluorophore) for the minimal deletion region in primary gliomas (GLTSCR1 maps within this BAC) and a control 19p probe (labeled with a green fluorophore). Seven of the 11 tumors exhibited 19q loss (or deletion) in all of the tumor cells or in an extensive region of tumor. There was no evidence of deletion in the remaining 4 tumors. Twenty-four tumor regions with and without loss were microdissected from 3 parallel 15 μm sections from each of these 11 oligodendrogliomas. Sixteen sections (at least one from each tumor) generated sufficient DNA for at least 50 independent Pyrosequencing™ reactions.

Of the 7 oligodendrogliomas with 19q (GLTSCR1) loss, 5 lost the GLTSCR1 G allele (e.g., the tumor became homozygous for the A allele) and 2 lost the A allele (e.g., the tumor became homozygous for the G allele). The 4 oligodendrogliomas without 19q loss maintained GLTSCR1 heterozygosity.

### Example 7

### Glioma Survival Risk and Correlations with SNP Alleles

Using the above case cohort, we have also compared the association of ERCC1-exon-1, ERCC2-exon-22, and GLTSCR1-exon-6 polymorphisms with patient survival and other clinical variables. Importantly, glioma patients with the GLTSCR1-exon-6 AA genotype had better survival rates: 73% and 61% survival at 2 and 5 years for the AA genotype compared to 45% and 17% at 2 and 5 years for the AG/GG genotype (p=0.01, log-rank test) (note that the “GLTSCR1-exon-6 AA genotype” refers to nucleotides located on the noncoding strands of the DNA chromosomes, and thus equates to a TT genotype with respect to the same positions on the coding strands of the chromosomes, i.e., at the nucleotide positions corresponding to position 1538 of SEQ ID NO:2). This significant difference in survival was also observed for the patients with oligodendrogliomas alone.

To identify subgroups with the longest and shortest survival, we used CART (LeBlanc and Crowley, Biometrics 48:411-425, 1992; Themeau and Atkinson, An introduction to recursive partitioning using the RPART routines. Department of Health Sciences Research, Section of Biostatistics, Technical Report#61, Mayo Clinic, Rochester, Minn., USA, 1997) modeling to determine clinical and genetic variables that were independently associated with survival. The CART model identified that grade, GLTSCR1 genotype, and age were the most informative variables for generating groups of glioma patients with similar survival experience. The 7 grade 2-3 gliomas with the GLTSCR1-exon-6 AA genotype had the best survival (hazard ratio=0.097; 95% CI undefined since no events). The 40 grade 4 glioma patients, who were 46 years of age, or older, had the worst survival (hazard ratio=3.2; 95% CI: 2.3 to 4.5). Morphology type was not selected by the model.

The ERCC1 and ERCC2 polymorphisms we tested were not significantly associated with glioma 19q deletion status, morphologic grade of glioma, or patient survival.

TABLE 4
Allele-Based Analysis of Association of Selected 19q SNPs with Glioma Development

| | General | All | | | | Mixed | | | |
|---|---|---|---|---|---|---|---|---|---|
| | Controls | Gliomas | | Astrocytomas | | Oligoastrocytomas | | Oligodendrogliomas | |
| | (N = 108) | (N = 143) | | (N = 61) | | (N = 40) | | (N = 42) | |
| Locus | n (%) | n (%) | p-value* | n (%) | p-value* | n (%) | p-value* | n (%) | p-value* |
| ERCC1 - Exon1 | 210 Chrs** | 280 Chrs | 0.514 | 118 Chrs | 0.909 | 80 Chrs | 0.314 | 82 Chrs | 0.673 |
| Allele A | 51 (24.3) | 61 (21.8) | | 28 (23.7) | | 15 (18.8) | | 18 (22.0) | |
| Allele C | 159 (75.7) | 219 (78.2) | | 90 (76.3) | | 65 (81.2) | | 64 (78.0) | |
| ERCC1 - Exon4 | 24 Chrs | 284 Chrs | 0.904 | 122 Chrs | 0.598 | 78 Chrs | 0.367 | 84 Chrs | 0.648 |
| Allele C | 78 (3605) | 105 (37.0) | | 48 (39.3) | | 24 (30.8) | | 33 (39.3) | |
| Allele T | 136 (63.5) | 179 (63.0) | | 74 (60.7) | | 54 (69.2) | | 51 (60.7) | |
| ERCC2 - Exon6 | 206 Chrs | 272 Chrs | 0.420 | 116 Chrs | 0.111 | 74 Chrs | 0.667 | 82 Chrs | 0.762 |
| Allele G | 109 (52.9) | 154 (56.6) | | 72 (62.1) | | 37 (50.0) | | 45 (54.9) | |
| Allele T | 97 (47.1) | 118 (43.4) | | 44 (37.9) | | 37 (50.0) | | 37 (45.1) | |
| ERCC2 - Exon22 | 210 Chrs | 274 Chrs | 0.827 | 120 Chrs | 0.821 | 76 Chrs | 0.418 | 78 Chrs | 0.093 |
| Allele A | 64 (30.5) | 81 (29.6) | | 38 (31.7) | | 27 (35.5) | | 16 (20.5) | |
| Allele G | 146 (69.5) | 193 (70.4) | | 82 (68.3) | | 49 (64.5) | | 62 (79.5) | |
| GLTSCR1 - Exon6 | 204 Chrs | 270 Chrs | 0.250 | 112 Chrs | 0.656 | 78 Chrs | 0.801 | 80 Chrs | 0.016 |
| Allele A | 50 (24.5) | 79 (29.3) | | 30 (26.8) | | 18 (23.1) | | 31 (38.8) | |
| Allele G | 154 (75.5) | 191 (70.7) | | 82 (73.2) | | 60 (76.9) | | 49 (61.2) | |
| LIG1 - Exon6 | 216 Chrs | 286 Chrs | 0.961 | 122 Chrs | 0.905 | 80 Chrs | 0.585 | 84 Chrs | 0.570 |
| Allele A | 103 (47.7) | 137 (47.9) | | 59 (48.4) | | 41 (51.3) | | 37 (44.1) | |
| Allele C | 113 (52.3) | 149 (52.1) | | 63 (51.6) | | 39 (48.7) | | 47 (55.9) | |
| RAI - Exon6 | 216 Chrs | 280 Chrs | 0.2445 | 118 Chrs | 0.173 | 80 Chrs | 0.742 | 82 Chrs | 0.171 |
| Allele A | 183 (84.7) | 226 (80.7) | | 93 (78.8) | | 69 (86.3) | | 64 (78.1) | |
| Allele T | 33 (15.3) | 24 (19.3) | | 25 (21.2) | | 11 (13.7) | | 18 (21.9) | |

*p-value = pearson's Chi-squared test (comparison to the Normal group)
**Chrs = Chromosomes

TABLE 5
Haplotype-Based Analysis of Association of Selected 19q SNPs with Oligodendroglioma Development

| ERCC1 | ERCC1 | ERCC2 | ERCC2 | GLTSCR1 | LTG1 | RAI | Empirical | Simulated | Haplotype Frequency | | |
|---|---|---|---|---|---|---|---|---|---|---|---|
| Exon1 | Exon4 | Exon6 | Exon22 | Exon6 | Exon6 | Exon6 | P-Value | P-Value | Overall | Controls | Cases |
| A | C | G | A | A | C | A | 0.86 | 0.86 | 0.015 | 0.014 | 0.037 |
| A | C | G | A | G | A | A | 0.056 | 0.048 | 0.074 | 0.095 | 0.000 |
| A | C | T | G | A | C | A | 0.64 | 0.63 | 0.027 | 0.026 | 0.053 |
| A | C | T | G | G | C | A | 0.29 | 0.31 | 0.052 | 0.043 | 0.000 |
| C | C | G | A | A | A | A | 0.0002 | 0.006 | 0.018 | 0.000 | 0.062 |
| C | C | G | G | G | C | A | 0.55 | 0.63 | 0.034 | 0.038 | 0.000 |
| C | T | G | A | G | A | A | 0.13 | 0.15 | 0.054 | 0.069 | 0.000 |
| C | T | G | A | G | C | A | 0.24 | 0.23 | 0.033 | 0.038 | 0.024 |
| C | T | G | G | A | A | T | 0.45 | 0.48 | 0.025 | 0.023 | 0.000 |
| C | T | G | G | A | C | T | 0.10 | 0.09 | 0.028 | 0.018 | 0.046 |
| C | T | G | G | G | A | T | 0.26 | 0.29 | 0.028 | 0.040 | 0.060 |
| C | T | G | G | G | C | T | 0.14 | 0.14 | 0.063 | 0.045 | 0.089 |
| C | T | T | G | A | A | A | 0.49 | 0.50 | 0.028 | 0.021 | 0.087 |
| C | T | T | G | A | C | A | 0.43 | 0.41 | 0.074 | 0.068 | 0.030 |
| C | T | T | G | G | A | A | 0.89 | 0.91 | 0.120 | 0.120 | 0.057 |
| C | T | T | G | G | C | A | 0.30 | 0.29 | 0.124 | 0.140 | 0.170 |

### Example 8

### Correlation of Prostate Cancer with GLTSCR1 Alleles

Using 161 high-risk pedigrees, an association between Gleason score and microsatellite markers on chromosome 19 was discovered (Slager et al., Am. J. Hum. Genet. 72:759-762, 2003). To compare genotype frequencies, including SNP frequencies, at candidate loci on chromosome 19 with high grade (grade>6) and low grade (grade<=6) tumors, a novel approach was developed to identify homogeneous populations for testing.

Using the chromosome 19 linkage data, a group of linkage brother pairs (i.e., brother pairs hypothesized to carry the chromosome 19 markers) was selected. A “linked” brother pair was defined as one in which the brothers were either (1) concordant for their disease status (as defined below) and with high mean ibd-sharing in the region (defined below), or (2) discordant for disease status and with low mean ibd-sharing in the region. “IBD” refers to “identity by descent”; two genes at a locus have ibd if they were both inherited from common ancestors.

For each brother pair, the mean ibd-sharing was computed using the multipoint ibd probabilities from MERLIN2. Five markers (D19S903, D19S412, D19S902, D19S879, D19S907) that surround the region showing strong linkage evidence were selected. For each of these markers, a cut point was used to define “high” or “low” mean ibd-sharing. The cut-off point for high mean ibd-sharing was 0.84, and for low mean ibd-sharing was 0.15. A brother pair that had high mean ibd-sharing across all five markers was considered as having “high mean ibd-sharing” in the region. Likewise, a brother pair that had low mean ibd-sharing across all five markers was considered as having “low mean ibd-sharing” in the region.

“Concordant for disease status” was defined as brothers with tumor grade within 2 units of each other and either both brothers having “low grade” (grade<=6) or both having “high grade” (grade>6) tumors. For example, a brother pair in which one brother was grade =4 and the other grade =6 was considered concordant for disease status. “Discordant for disease status” meant that the brother pairs were not concordant for disease. For example, a brother pair was discordant if one brother was grade =6 and the other was grade=7 because the two brothers fell into separate disease categories.

Combining the ibd information and the disease concordance for each pair, the set of pairs that met the criteria were selected as being linked at the chromosome 19 region. These pairs made up the homogeneous subgroup of cases that was used to test for an association with candidate-gene loci. The Armitage test for trend with a variance correction for related individuals was used for this study (Slager and Schaid, Am. J. of Hum. Genet. 68:1457-1462, 2001). Empirical p-values were obtained based on 5,000 simulations. The original genotypes were retained and the high/low grade disease categories were permuted among the subset of subjects within each family. The empirical p-value was the proportion of times a calculated result was more significant than the observed result.

Out of the possible 193 brother pairs selected from 161 high-risk families, 32 satisfied the criteria for being “linked” at the chromosome 19 region. Seven brother pairs had low mean ibd-sharing and discordant Gleason scores. The remaining pairs were concordant for disease and had high mean ibd-sharing. The 32 brother pairs came from 30 different families (one family consisted of three affected brothers) and formed the homogeneous subgroup of 61 cases used to test for association with the twelve candidate-gene loci. The cases were categorized into high or low Gleason score. Forty-six cases had low grade scores (grade<=6) and 15 cases had high grade scores (grade>6). The fifteen high-grade cases came from 11 distinct families, and the 46 low-grade cases came from 26 distinct families.

The GLTSCR1 gene from a subset of the selected group of men was sequenced. This subset included 48 men, including 16 men with a high Gleason score (i.e., high grade) and 32 men with a low Gleason Score (i.e., low grade). Full length GLTSCR1 mRNA was first compared with genomic DNA. A total 15 exons were identified, 13 of which were protein-coding. Because the 13 protein-coding exons were extremely GC-rich, and the conventional technique for mutation screening would not be effective for these types of DNA sequences, a PCR-direct sequencing technique was used for mutation detection. Twenty pairs of PCR primers were designed to amplify the 13 exons and their flanking regions. The PCR primers and conditions are listed in Table 6. All exons except exon 8 were amplified using the following PCR conditions: 35 cycles with initial denaturation at 95° C. for 15 min, followed by 94° C. for 50 sec, 55-60° C. for 1 min and 72° C. for 1 min. The reaction was processed in a total volume of 15 μl consisting of 200 μM of each dNTP, 0.25 μM of each of PCR primers, 1.5 mM of MgCl₂, 30 ng of template DNA, 1× HotStarTaq buffer, 1× Q solution and 0.1 unit of HotStarTaq DNA polymerase (QIAGEN, Valencia, Calif.). Exon 8 was amplified using the same conditions, except GoTaq DNA polymerase (Promega, Madison, Wis.) was used instead of HotStarTaq. Five microliters of the PCR products were treated with 1 μl (10.0 units) Exonuclease I and 1 μl (2.0 units) Shrimp Alkaline Phosphatase (USB Corp., Cleveland, Ohio) at 37° C. for 15 min and 80° C. for 15 min. The treated products were then diluted in a ratio of 1 to 4. Three microliters of the phosphatased PCR product and 1.6 pmol of corresponding PCR primer were mixed and sequenced at the Molecular Core Facility at Mayo Clinic on an ABI PRISM 3700 DNA Analyzer. All PCR products were sequenced twice (once forward and once in reverse).

TABLE 6
PCR primers used in GLTSCR1 gene sequencing

| | | | | |
|---|---|---|---|---|
| | Primer | | PCR | Annealing |
| Exons | Names | Primer sequences (5′′3′) | sizes | Temperatures |

| | | | | |
|---|---|---|---|---|
| 3 + 4 | lw856 | GGCATGGTCAGGACTCACTTAG | 559 bp | 55° C. |
| | lw857 | AGTGCCTGGCGTTAGAAGC | | |
| 5 | lw858 | ATTCGAGGCAGTCACAGCAG | 363 bp | 58° C. |
| | lw859 | CCAACAGCTCAGGCAATGAT | | |
| 6 | lw829 | AGTGCTAAGATTACAGGCGTGAGTC | 462 bp | 60° C. |
| | lw832 | TGAGGTAGGAGGATGGATGGC | | |
| 6 | lw831 | CTAAGACCACAGGCACATGC | 336 bp | 55° C. |
| | lw830 | TTGGGCTCTGGGTTCAGGTGGTTGC | | |
| 6 | lw809 | TTCCGGCAACCACCTGAA | 405 bp | 60° C. |
| | lw810 | GCCTTGTTGACCACGTCCT | | |
| 6 | lw811 | CAACATCACGGAGCAGACG | 551 bp | 60° C. |
| | lw812 | CCGAGAGGTTCTTCTGGATGAC | | |
| 6 | lw835 | TCATCCAGAAGAACCTCTCG | 816 bp | 55° C. |
| | lw836 | ACACGGGCATCTGGAAGAGC | | |
| 6 | lw813 | GTGGACAGCTCATCGCGAAC | 725 bp | 55° C. |
| | lw814 | TGCCATGACTGAGACCTTCC | | |
| 7 | lw815 | AACCCTGGGAGCGTCAATAG | 382 bp | 55° C. |
| | lw816 | GTCAGCAGAACAGAGACGCA | | |
| 8 | lw847 | GTGTCTTGGGGCAACAGGAGCTATG | 752 bp | 58° C. |
| | lw848 | TCCCATCAGACTGAGAGGGTGGGAT | | |
| 9 | lw837 | TTCCAGATGGTGACCACCCCCTT | 556 bp | 55° C. |
| | lw838 | TGTGTCCCTTGAGTTCTGGC | | |
| 10 | lw849 | AACTCAAGGGACACAGGAAGGC | 483 bp | 62° C. |
| | lw850 | GTGTTTCTCCCTCATCTCTGGCT | | |
| 11 | lw839 | TATCCTAGGCTAGAGGGGAAGC | 290 bp | 55° C. |
| | lw840 | CTATGAGGGAACTACATCGC | | |
| 12 | lw841 | CTCAGTGAGATGGAACAAGC | 522 bp | 55° C. |
| | lw842 | TCAAACTCCTCGTCCACTGC | | |
| 13 | lw821 | CAGGTCCACGGTGCGCTAT | 415 bp | 55° C. |
| | lw822 | CGAGTGAACCAGCAAGAAGG | | |
| 14 | lw843 | TCACAAGGACAGTTTGGACC | 282 bp | 55° C. |
| | lw844 | CGCCCTGAATCTAACTCTTC | | |
| 15 | lw823 | TCCCCATCGCAGCCTCTT | 556 bp | 55° C. |
| | lw824 | ACGTTCTCGCGGTAGGTCT | | |
| 15 | lw825 | AGATGAACGGCACGGTGGA | 458 bp | 55° C. |
| | lw826 | GTTCAGGATGCTGTCGATGG | | |
| 15 | lw827 | CGAGCTGTACCAGCGTATGC | 455 bp | 55° C. |
| | lw828 | GTCCCCAGGAGGCTGAGG | | |
| 15 | lw845 | AGGTAGTAGGTGCTCACTGC | 247 bp | 55° C. |
| | lw846 | TGCCGGATCACAAGCTTGGT | | |

The most common SNP identified had a carrier odds ratio of 11.67 (95% CI: 2.76,49.41). This SNP is 852 kb from the microsatellite marker that had the highest LOD score (D19S902) in the linkage analysis. Table 7 shows the genotype distribution across test subjects. Three other variants from this gene were then genotyped and tested for association with this subgroup of cases (Tables 8-10). The four SNPs from the GLTSCR1 gene were within 20 kb of each other and showed strong evidence for linkage disequilibrium (D′=1 for all pairs). Four different SNPs (two located on each side of the GLTSCR1 gene) from an Affymetric 10K SNP chip were tested for similar associations. These SNPs are not located in any known gene and none were found to be significant.

TABLE 7
Genotype counts (frequency) for rs1035938 SNP (Exon 6
1344C > T of Table 11^{a}) in GLTSCR1 gene

| | WW* | WV | VV | Total |
|---|---|---|---|---|
| High grade | 5 (0.33) | 9 (0.60) | 1 (0.07) | 15 |
| Low grade | 35 (0.85) | 6 (0.15) | 0 (0) | 41 |

W = most frequent allele
Armitage's Trend Test: χ² = 11.9, df = 1, Permutation P = 0.0006
Carrier Odds Ratio = 11.67 (95% CI: 2.76, 49.41)
^{a}Nucleotide 1344 of Table 11 corresponds to nucleotide 1538 in SEQ ID NO: 2

TABLE 8
Genotype counts (frequency) for rs3745762 SNP (Exon 6
2047C > T of Table 11^{a}) in GLTSCR1 gene

| | WW* | WV | VV | Total |
|---|---|---|---|---|
| High grade | 5 (0.33) | 9 (0.60) | 1 (0.07) | 15 |
| Low grade | 35 (0.85) | 6 (0.15) | 0 (0) | 41 |

W = most frequent allele
Armitage's Trend Test: χ² = 12.0, df = 1, Permutation P = 0.0004
Carrier Odds Ratio = 11.67 (95% CI: 2.83, 48.17)
^{a}Nucleotide 2047 of Table 11 corresponds to nucleotide 2241 in SEQ ID NO: 2

TABLE 9
Genotype counts (frequency) for SNP rs2914430 (G > A)
in intron 7 of the GLTSCR1 gene

| | WW* | WV | VV | |
|---|---|---|---|---|
| | GG | GA | AA | Total |
| High grade | 6 (0.40) | 7 (0.47) | 2 (0.13) | 15 |
| Low grade | 9 (0.20) | 17 (0.39) | 18 (0.41) | 44 |

W = most frequent allele
Armitage's Trend Test: χ² = 3.19, df = 1, Permutation P = 0.078
Carrier Odds Ratio = 0.39 (95% CI: 0.094, 1.577)

TABLE 10
Genotype counts (frequency) for SNP rs1005911
(T > G) 3′ of the coding region of the GLTSCR1 gene

| | WW* | WV | VV | |
|---|---|---|---|---|
| | TT | TG | GG | Total |
| High grade | 4 (0.29) | 9 (0.64) | 1 (0.07) | 14 |
| Low grade | 35 (0.81) | 8 (0.19) | 0 (0.0) | 43 |

W = most frequent allele
Armitage's Trend Test: χ² = 11.2, df = 1, Permutation P = 0.0008
Carrier Odds Ratio = 10.94 (95% CI: 2.451, 48.803)

### Example 9

### Sequence of GLTSCR1 and Association with Prostate Cancer

Forty-eight familial prostate cancer patients were sequenced for potential germline mutation within the GLTSCR1 gene. A total of 38 variants were identified (Table 11). Among these variants, 20 were intronic and 18 exonic. Of the 18 exonic changes, 17 were located in protein-coding sequence and included 7 missense and 10 silent alterations (Table 11). Among the 7 missense mutations, two (683P→S and 1044T→A) were common polymorphisms. These shared an identical allele and were previously reported in the SNP database (rs3745762 and rs13346368). One of these was tested previously (Table 8). The remaining five missense changes (384P→A, 683P→S, 825P→L, 863R→C and 1475R→H) were rare mutations. These rare mutations were too infrequent to statistically test for an association with Gleason score.

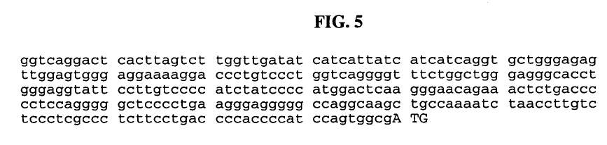

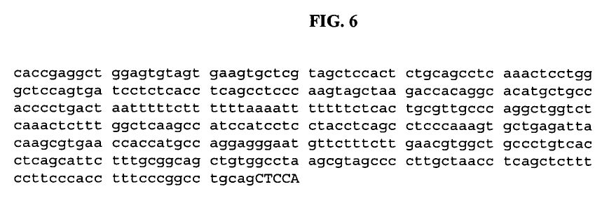

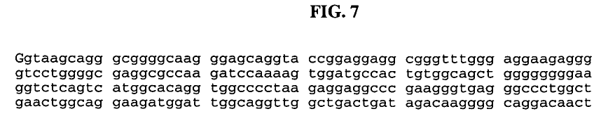

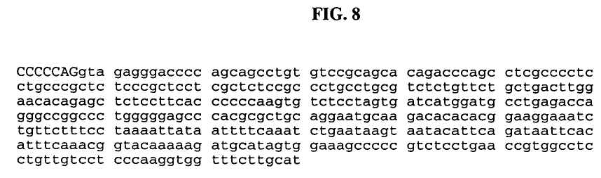

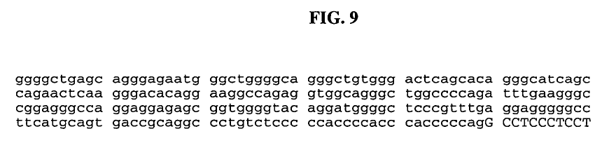

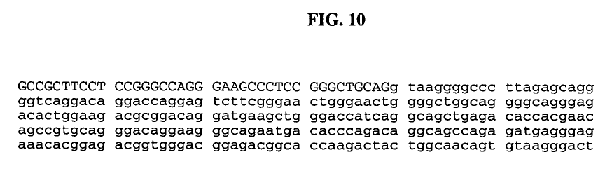

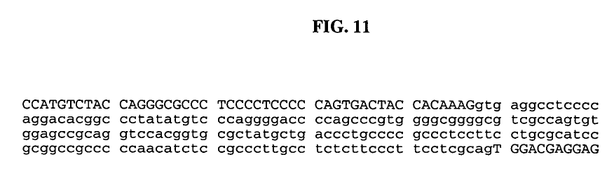

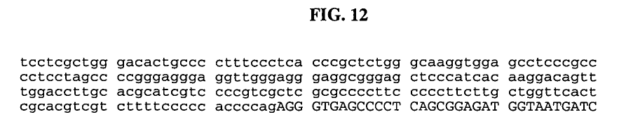

TABLE 11
GLTSCR1 gene sequencing in 48 patients with Prostate Cancer

| | | Amino Acid | | |
|---|---|---|---|---|
| | | Changes | | |
| | | (missense | | |
| Exon/ | Nucleotide | multations in | Genomic | Minor Allele |
| Intron | Changes^{a} | bold) | Location | Frequency (%) |
| Intron 2 | 1-249C > A | | 65131 | 1.04 |
| | 1-147T > C | | 65233 | 1.04 |
| Intron 5 | 151-242T > A | | 70884 | 3.13 |
| | 151-207C > T | | 70919 | 12.50 |
| | 151-70G > A | | 71056 | 46.88 |
| | 151-56A > G | | 71070 | 17.71 |
| | 151-10T > C | | 71116 | 26.04 |
| Exon 6 | 360C > T | 120A > A | 71335 | 17.71 |
| | 492C > T | 164T > T | 71467 | 1.04 |
| | 519C > T | 173P > P | 71494 | 26.00 |
| | 687G > A | 229T > T | 71662 | 26.00 |
| | 1150C > G | 384P > A | 72125 | 1.04 |
| | 1344C > T | 448S > S | 72319 | 17.71 |
| | 1428C > T | 476G > G | 72403 | 2.08 |
| | 1574G > A | 525S > N | 72549 | 1.04 |
| | 1632C > T | 544A > A | 72607 | 1.05 |
| | 1845C > T | 615A > A | 72820 | 1.05 |
| | 2047C > T | 683P > S | 73022 | 17.89 |
| Intron 6 | 2106 + 14A > G | | 73095 | 44.68 |
| | poly(G)7-8 | | 73190 | 15.63 |
| Intron 7 | 2283 + 80C > G | | 74037 | 18.75 |
| Exon 8 | 2474C > T | 825P > L | 86110 | 2.08 |
| | 2587C > T | 863R > C | 86223 | 1.00 |
| Intron 9 | 3077 −65A > G | | 87105 | 48.89 |
| Exon 10 | 3130A > G | 1044T > A | 87223 | 15.56 |
| Intron 10 | 3186 +65G > C | | 87344 | 40.00 |
| | 3186 +86A > T | | 87365 | 26.67 |
| | 3186 +93C > T | | 87372 | 2.22 |
| | 3186 +102C > G | | 87381 | 44.44 |
| | 3186 +152G > A | | 87431 | 2.22 |
| Intron 12 | 3397 +63G > T | | 90650 | 1.04 |
| | 3397+86G > C | | 90673 | 26.04 |
| | 3398−44C > T | | 90726 | 2.08 |
| Intron 13 | 3493−62C > T | | 91046 | 8.33 |
| Exon15 | 4299G > A | 1433A > A | 93836 | 5.21 |
| | 4424G > A | 1475R > H | 93961 | 1.04 |
| | 4659C > A | 1553L > L | 94196 | 1.04 |
| Exon 15 | 4683+53T > C | | 94273 | 19.79 |
| (5′ UTR) | | | | |
| ^{a}Nucleotides are numbered according to the full-length protein-coding mRNA sequence, “1” being the adenine in the start codon (ATG) of the GLTSCR1 mRNA, and corresponding to nucleotide 195 in SEQ ID NO: 2 (FIG. 2). Intron nucleotide changes are designated with reference to the numbering of the nucleotides in the full-length protein-coding mRNA. For example, in intron 1, the nucleotide change is 249 nucleotides 5′ of the | | | | |
| # adenine of the ATG start codon. In Exon 15, the nucleotide change is 53 nucleotides 3′ of nucleotide 4683 of the GLTSCR1 mRNA (nucleotide 4683 corresponds to nucleotide 4877 of SEQ ID NO: 2). Intron sequences are shown in FIGS. 5-12. | | | | |

For the intronic variants (Table 11), a mononucleotide repeat (G)7-8 was identified in 15 of the 48 patients. The repeat was located at intron 6, 109 bp downstream of exon 5. The remaining variants were single nucleotide substitution (Table 11).

To further evaluate the frequency of these rare alleles, 48 individuals without prostate cancer were examined for the presence of three of the missense changes. We did not detect these variant alleles (384P→A, 683P→S and 1475R→H) in any of these normal controls.

### Other Embodiments

A number of embodiments of the invention have been described. Nevertheless, it will be understood that various modifications may be made without departing from the spirit and scope of the invention. Accordingly, other embodiments are within the scope of the following claims.

1. A method for diagnosing a patient as having an elevated risk of developing a proliferative disorder, comprising:
(i) providing a biological sample from a patient;
(ii) performing a genetic analysis of a GLTSCR1 nucleic acid from said biological sample; and
(iii) if a genetic abnormality is detected in said GLTSCR1 nucleic acid, determining that said patient is at risk for developing a proliferative disorder.

2. The method of claim 1, wherein said proliferative disorder is a glioma.

3. The method of claim 2, wherein said glioma is an oligodendroglioma.

4. The method of claim 1, wherein said proliferative disorder is prostate cancer.

5. The method of claim 1, wherein said biological sample is a blood sample.

6. The method of claim 1, wherein said genetic analysis comprises a deletion mapping study.

7. The method of claim 1 wherein said genetic analysis comprises FISH, homozygosity mapping, cytogenetics, spectral karyotyping, or comparative genomic hybridization to arrays.

8. The method of claim 1, wherein said genetic analysis comprises a SNP analysis.

9. The method of claim 1, wherein said genetic abnormality is a deletion mutation.

10. The method of claim 9, wherein said deletion mutation comprises at least a fragment of exon 6 of said GLTSCR1 nucleic acid.

11. The method of claim 1, wherein said genetic abnormality comprises a SNP in exon 6 of said GLTSCR1 nucleic acid.

12. The method of claim 11, wherein said SNP in exon 6 comprises a T at the position corresponding to position 1538 of SEQ ID NO:2.

13. The method of claim 1, further comprising determining that said patient is homozygous for a C at the position corresponding to position 1538 of SEQ ID NO:2, and determining that said patient does not have an elevated risk of developing a glioma.

14. The method of claim 1, further comprising notifying said patient, or a caregiver of said patient, of said diagnosis.

15. The method of claim 1, further comprising recording said diagnosis in print or in a computer-readable format.

16. A method for determining a prognosis for a glioma patient comprising:
(i) providing a biological sample from said patient;
(ii) performing a SNP analysis of the section of chromosome 19q comprising the nucleotide position corresponding to position 1538 of SEQ ID NO:2 of said glioma sample; and
(iii) determining that said patient has (A) a good prognosis if the patient is homozygous or hemizygous for a T at the position corresponding to position 1538 of SEQ ID NO:2, or (B) a baseline prognosis if the patient is heterozygous, hemizygous, or homozygous for a C at the position corresponding to position 1538 of SEQ ID NO:2.

17. The method of claim 16, wherein said biological sample is a glioma sample.

18. The method of claim 16, wherein said SNP analysis comprises FISH, homozygosity mapping, cytogenetics, spectral karyotyping, or comparative genomic hybridization to arrays.

19. The method of claim 16, further comprising notifying said patient, or a caregiver of said patient, of said prognosis.

20. The method of claim 16, further comprising recording said prognosis in print or in a computer-readable format.

21. A method for diagnosing a patient as having an elevated risk of developing a prostate cancer, comprising:
(i) providing a biological sample from a patient;
(ii) performing a genetic analysis of chromosome 19q from said biological sample; and
(iii) detecting a SNP at one or more of nucleotide positions 1344, 1538, 1768, 2241, 2668, 2781, 3324, and 4618 as defined by SEQ ID NO:2, wherein the presence of a SNP indicates that said patient has an elevated risk of developing a prostate cancer.

22. A kit comprising reagents and instructions for determining the genotype of a human at chromosome 19q, and determining said human's risk of developing a proliferative disorder or said human's prognosis for surviving said proliferative disorder.

23. The kit of claim 22, comprising reagents for FISH or comparative genomic hybridization to arrays.

24. The kit of claim 22, wherein said proliferative disorder is a glioma.

25. The kit of claim 24, wherein said glioma is an oligodendroglioma.

26. The kit of claim 22, wherein said proliferative disorder is a prostate cancer.
